(19) United States
(12) Patent Application Publication
(10) Pub. No.: US 2006/0292574 A1
(43) Pub. Date: Dec. 28, 2006

(54) Automated VNTR genotyping method

(75) Inventors: Ruwen Jou, Taipei City (TW); Pei-Ju Chin, Taipei City (TW)

Correspondence Address: BIRCH STEWART KOLASCH & BIRCH, PO BOX 747, FALLS CHURCH, VA, 22040-0747, US

(73) Assignee: Center for Disease Control, Department of Health, Taiwan, R.O.C.

(21) Appl. No.: 11/137,334

(22) Filed: May 26, 2005

(51) Int. Cl. C12Q 1/68; C12P 19/34

(52) U.S. Cl. 435/6; 435/91.2

(57) ABSTRACT

The invention provides an automated VNTR genotyping method using multiple variable-number tandem repeats (VNTRs) loci based on mycobacterial interspersed repetitive units (MIRU) undergoing multiplex PCR and high throughput MegaBACE® capillary electrophoresis system. The method uses fluorescent dyes of 6-carboxytetramethylrhodamine(TAMRA), 6-carboxy fluorescein (FAM) and 6-carboxy-2′,4,4′,5′,7,7′-hexachlorofluorescein (HEX) labeling PCR primers. The method results in an efficient VNTR genotyping with low cost, less labor-requirement and less reaction time. The method is applicable in organism analyses by VNTR genotyping, such as microorganisms, parasites, animals or plants.

### BACKGROUND

The invention relates to an automated VNTR genotyping method.

Mycobacterium tuberculosis (M. tuberculosis), which causes Tuberculosis (TB), threatens about 10 million people worldwide every year and causes nearly 3 million deaths. An efficient method for discriminating various strains of M. tuberculosis has long been sought after. M. tuberculosis has a variety of tandem-repeat loci on chromosomes discovered in 1998. Different strains have various repeat numbers at different loci resulted in polymorphism. Therefore, polymorphism of VNTR has been considered a means of distinguishing strains of M. tuberculosis.

Since 1993, restriction fragment length polymorphism (RFLP) has been used to type M. tuberculosis by targeting IS6110 (an insertion sequence), a transposable element. An adequate discriminatory power has been obtained. However, RFLP analysis is labor-intensive and difficult to reproduce thus hindering comparison between laboratories. It has been found that IS6110 RFLP analysis does not discriminate well in low-copy-number of M. tuberculosis (Cowan L. S. et al., 2002, J Clin Microbiol 40:1592-1602).

Richard F. and Winifred A. M. have provided a molecular genotyping method which use variable number tandem repeats (VNTRs) as genomic markers. The report revealed that the lengths of amplicons obtained from PCR can serve as the basis for M. tuberculosis genotyping method that requires less time and labor than conventional RFLP (Richard F & Winifred A. M., 1998, Microbiology 144:1189-1196).

Spoligotyping is also a well-estabilished genotyping method based on spacer sequences found in the direct repeat region in the M. tuberculosis chromosome. Although spoligotyping is less time-consuming and expensive, the result has poor discriminatory power (Skuce R. A. et al., 2002, Microbiology 148:519-528).

Philip Supply et al provided a genotyping method of VNTR of tuberculosis based on mycobacterial interspersed repetitive units (MIRU), and they intended to render multiple molecular genotyping practicability by multiplexing PCR and capillary electrophoresis (Supply P. et al., 2001, J. Clin. Microbiol. 39:3563-3571).

Allix C. et al used a MIRU-VNTR genotyping in clinical mycobacteriological analysis by adaptation and optimization of MIRU-VNTR genotyping on a capillary electrophoresis system. They used 4 sets of multiplex PCRs amplifying 12 loci of M. tuberculosis and then isolated the resulting DNA fragments by ABI Prism Genetic Analyzer (Applied Biosystems). The result was concordant to that genotyped by IS6110 RFLP. The MIRU-VNTR typing technique was faster and accurately resolved problems commonly encountered in clinical mycobacteriology (Allix C. et al., 2004, Clin. Infec. Dis. 39:783-789).

### SUMMARY

An automated VNTR genotyping method is provided by amplifying the tandem repeat loci by a polymerase chain reaction (PCR) and encoding each locus in numerals based on estimated repeat numbers obtained from PCR products.

An embodiment of the automated VNTR genotyping method comprises obtaining a DNA fragment from a sample of interest; amplifying the DNA fragment by a PCR reaction; detecting the amplified DNA fragment by an automated system. The PCR reaction uses primers labeled by fluorescent dyes selected from the group of TAMRA, FAM and HEX.

Another embodiment of the automated VNTR genotyping method further comprises obtaining a DNA fragment from a sample of interest; amplifying the DNA fragment by a PCR reaction; detecting the amplified DNA fragment by an automated capillary electrophoresis system. The PCR reaction uses primers labeled by fluorescent dyes selected from the group of TAMRA, FAM and HEX.

Another embodiment of the automated VNTR genotyping method comprises obtaining a DNA fragment from a sample of interest; amplifying the DNA fragment by a PCR reaction; detecting the amplified DNA fragment by an automated capillary electrophoresis system. The PCR reaction uses 15 primers (Table 1) labeled by fluorescent dyes selected from the group of TAMRA, FAM and HEX.

The capillary electrophoresis system can be MegaBACE® capillary electrophoresis system (GE-Amersham Bioscience).

The automated VNTR genotyping method of the invention can more rapidly obtain a discriminatory power better than conventional RFLP methods with lower cost, less labor-consuming and less reaction time.

### DETAILED DESCRIPTION

The invention provides an automated VNTR genotyping method comprising obtaining a DNA fragment from a sample of interest; amplifying the DNA fragment by a PCR reaction; detecting the amplified DNA fragment by an automated system. The PCR reaction uses primers labeled by fluorescent dyes selected from the group of TAMRA, FAM and HEX.

The automated VNTR genotyping method can further comprise an automated capillary electrophoresis system.

The automated capillary electrophoresis system can be MegaBACE® capillary electrophoresis system (GE-Amersham Bioscience).

The automated VNTR genotyping method is able to genotype microorganism, parasites, animals, or plants, particularly M. tuberculosis.

The automated VNTR genotyping method can use primers in the PCR reaction for MIRU-VNTR genotyping as the following:

TABLE 1

Primer sets for multiplex PCR of
MIRU-VNTR analysis

| Multiplex | | VNTR | |
|---|---|---|---|
| PCR | | Length | Primer sequence |
| mixture | Locus^{a} | (bp) | (labeling) (5′-3′)^{b} |
| Panel-A | 4 | 77 | (FAM)GCGCGAGAGCCCGAACTGC |
| | (ETR-D) | | GCGCAGCAGAAACGCCAGC |
| | 26 | 51 | TAGGTCTACCGTCGAAATCTGTGAC |
| | | | (HEX)CATAGGCGACCAGGCGAATA |
| | | | G |
| | 40 | 54 | (TAMRA)GGGTTGCTGGATGACAAC |
| | | | GTGT |
| | | | GGGTGATCTCGGCGAAATCAGATA |
| Panel-B | 10 | 53 | GTTCTTGACCAACTGCAGTCGTCC |
| | | | (FAM)GCCACCTTGGTGATCAGCTA |
| | | | CCT |
| | 16 | 53 | TCGGTGATCGGGTCCAGTCCAAGTA |
| | | | (HEX)CCCGTCGTGCAGCCCTGGTA |
| | | | C |
| | 31 | 53 | ACTGATTGGCTTCATACGGCTTTA |
| | (ETR-E) | | (TAMRA)GTGCCGACGTGGTCTTGA |
| | | | T |
| Panel-C | 2 | 53 | TGGACTTGCAGCAATGGACCAACT |
| | | | (FAM)TACTCGGACGCCGGCTCAAA |
| | | | AT |
| | 23 | 53 | (HEX)CTGTCGATGGCCGCAACAAA |
| | | | ACG |
| | | | AGCTCAACGGGTTCGCCCTTTTGTC |
| | 39 | 53 | CGCATCGACAAACTGGAGCCAAAC |
| | | | (TAMRA)CGGAAACGTCTACGCCCC |
| | | | ACACAT |
| Panel-D | 20 | 77 | (FAM)TCGGAGAGATGCCCTTCGAG |
| | | | TTAG |
| | | | GGAGACCGCGACCAGGTACTTGTA |
| | 24 | 54 | CGACCAAGATGTGCAGGAATACAT |
| | | | (HEX)GGGCGAGTTGAGCTCACAGA |
| | | | A |
| | 27 | 53 | TCGAAAGCCTCTGCGTGCCAGTAA |
| | | | (TAMRA)GCGATGTGAGCGTGCCAC |
| | | | TCAA |
| Panel-E | ETR-A | 75 | (FAM)AAATCGGTCCCATCACCTTC |
| | | | TTAT |
| | | | CGAAGCCTGGGGTGCCCGCGATTT |
| | ETR-B | 57 | (HEX)GCGAACACCAGGACAGCATC |
| | | | ATG |
| | | | GGCATGCCGGTGATCGAGTGG |
| | ETR-C | 58 | GTGAGTCGCTGCAGAACCTGCAG |
| | | | (TAMRA)GGCGTCTTGACCTCCACG |
| | | | AGTG |

^{a}Locus designations are according to the position (in kilobase paris) on the M. tuberculosis chromosome. VNTRs 4 and 31 correspond to the designations of ETR-D and EDR-E, respectively.
^{b}Labeling is indicated in the parentheses: FAM, 6-carboxy fluorescein; HEX, 6-carboxy- 2′, 4, 4′, 5′, 7, 7′-hexachlorofluorescein; TAMRA, 6-carboxytetramethylrhodamine.

The invention further provides an automated VNTR genotyping method, comprising obtaining a DNA fragment from a sample of interest; amplifying the DNA fragment by a PCR reaction; detecting the amplified DNA fragment by an automated capillary electrophoresis system. The PCR reaction uses primers labeled by fluorescent dyes selected from the group of TAMRA, FAM and HEX.

The automated capillary electrophoresis system can be MegaBACE® capillary electrophoresis system (GE-Amersham Bioscience).

The automated VNTR genotyping method is able to genotype microorganism, parasites, animals, or plants, particularly M. tuberculosis.

According to the invention, applicable fluorescent dyes are TAMRA (6-carboxytetramethylrhodamine), FAM (6-carboxy fluorescein) and HEX (6-carboxy-2′,4,4′,5′,7,7′-hexachlorofluorescein). These dyes are cheaper than the patented dye 2,7′,8′-benzo-5′-fluoro-2′,4,7-trichloro-5-carboxyfluorescein (NED®, ABI®) which is conventionally used with ABI Prism Genetic Analyzer (Applied Biosystems), and do not adversely affect the resultant discriminatory power.

The fluorescent dyes label the 15 primers employed by the invention to detecting the corresponding 15 loci of MIRU-VNTR (Table 1), in which the 12 primers are from Supply P. et al (Supply P. et al., 2001, J. Clin. Microbiol. 39:3563-3571), herein incorporated in its entirety. The additional 3 primers (ETR-A, ETR-B, ETR-C) multiplex Panel E listed in Table 1, which can enhance discriminatory power.

The amplified DNA fragments are then isolated by MegaBACE® capillary electrophoresis system (GE-Amersham Bioscience). The MegaBACE® capillary electrophoresis system is a high-throughput, fluorescent-based DNA system utilizing capillary electrophoresis with a maximum of 96 capillaries operating in parallel. The system performs sample injection, gel matrix replacement, DNA separation, detection, and data analysis. Due to automation, the MegaBACE® capillary electrophoresis system is able to speed up the genotyping of the invention and prevent the occurrence of artificial errors compared to RFLP. Additionally, the MegaBACE® capillary electrophoresis system is less expensive than the conventional ABI® system (Applied Biosystems).

The automated VNTR genotyping method can result in not only low cost but reduce time requirements. Compared to conventional RFLP and spoligotyping, MIRU-VNTR is able to type 75 stains per day, approximately 7 times more than RFLP does, but requires only about one tenth the cost of RFLP (Table 2). Moreover, the total operation time of RFLP takes approximately 7 days, 2 days for spoligotyping, and only 4 hours for MIRU-VNTR of the invention (data not shown). The required amount of PCR reagents, such as dNTP, Taq polymerase and final PCR products, are reduced to a fifth of that required by the conventional RFLP typing methods (see Table 5).

TABLE 2
Cost analyses of different genotyping methods
performed in the TB reference Lab of the CDC, Taiwan

| | RFLP | Spoligotyping | MIRU-VNTR |
|---|---|---|---|
| Special | Gel | Mini blotter | Capillary |
| instrument | electrophoresis | set | electrophoresis |
| requirement | system | | system |
| Capacity per | ˜10 | ˜20 | ˜75 |
| day | | | |
| (strains) | | | |
| Cost¹ | ˜8 | ˜5 | ˜0.7 |

Note:
¹cost shown in USD (TWD:USD = 31.15:1)

The following definitions of the specification are used herein:

“VNTR” refers to a variable number tandem repetitive unit in genomic DNA, usually used in a genotyping method.

“MIRU” refers to a mycobacterial interspersed repetitive unit of genomic DNA from tuberculosis, used in genotyping M. tuberculosis strains.

“MIRU-VNTR” refers to a VNTR genotyping of M. tuberculosis based on MIRUs.

“PCR” is a polymerase chain reaction for rapid amplifying DNA fragments in molecular research.

“Multiplex PCR” is a technique for simultaneous amplifying multiple loci in one PCR reaction.

“Genotyping” is a process of analyzing a particular genetic variation (polymorphism) existing in an individual DNA sample.

“Polymorphism” refers to a variety of tandem repeat loci in a population genome. It is usually used in population genetics to distinguish strains of organisms.

“RFLP” refers to restriction of fragment length polymorphism, which is a genotyping technique based on measuring the number and length of specific DNA fragments that are cut using specific restriction enzymes. The RFLP technique used to genotyping M. tuberculosis is based on the IS6110 insertion sequence.

“Capillary electrophoresis” is a family of techniques for efficiently separating a variety of compounds, performing in a narrow column driven by electric power.

“MegaBACE® capillary electrophoresis system” is an automated capillary electrophoresis system commercially available from GE-Amersham Bioscience, which comprises a capillary array, a capillary electrophoresis instrument, a separation matrix for DNA analysis, reagents for DNA sequencing and genotyping, instrument control and data analysis software, a computer workstation and monitor for instrument operation and data analysis. The system can perform sample injection, gel matrix replacement, DNA separation, detection, and data analysis.

“Discriminatory power” is an average probability that the typing system will assign a different type to two unrelated strains randomly sampled in the microbial population of a given taxon. The calculation of the invention is provided in the Example provided herein.

“HGDI” refers to Hunter-Gaston discriminatory index presenting discriminatory power. The calculation is based on Robin A. S. et al., Microbiology, 2002, 148, 519-528, herein incorporated in its entirety.

“Fluorescent dyes” are used to label DNA fragments in analyses. According to the invention, “TAMRA” is carboxytetramethylrhodamine, attaching to amino-modified oligonucleotides as a reporter dye; “HEX” is 6-carboxy-2′, 4,4′,5′,7,7′-hexachlorofluorescein added to the 5′-end of oligonucleotides; and “FAM” is 6-carboxyfluorescein attaching to 3′- or 5′-end of oligonucleotides. Three of the fluorescent dyes, according to the invention, are inexpensive enough to reduce costs when compared to conventional methods.

The aim of the invention is to develop an economically automated VNTR genotyping and a new application of the MegaBACE® capillary electrophoresis system (GE-Amersham Bioscience) for DNA typing, thereby improving the cost and labor-requirements of the system, and feasible for adaptation in high-throughput genotyping.

### EXAMPLE

—Fluorescent Labeled PCR Primer—

12 PCR primers were adapted from Supply P. et al (2001) with the exception that the MIRU 4 reverse primer was changed to 5′-GCG CAG CAG AAA CGC CAG C-3′ (Table 1). Three additional PCR primers set to produce ETR-A, ETR-B, and ETR-C amplicons were assigned to multiplexing Panel-E (Table 1). All fluorescent dyes were duly labeled at their 5′ ends, whereas the conventional used 2,7′,8′-benzo-5′-fluoro-2′, 4,7-trichloro-5-carboxyfluorescein (NED®) dye was replaced with 6-carboxytetramethylrhodamine(TAMRA) in this study. Aside from TAMRA, 6-carboxy fluorescein (FAM) and 6-carboxy-2′,4,4′,5′,7,7′-hexachlorofluorescein (HEX) were used for each multiplex. Five multiplex PCRs amplification of the 15 primers were used.

—PCR Reaction—

Each PCR reaction contains:

| Reagent | Volume (μl) |
|---|---|
| Sterilized deionized H₂O | 4.24(4.16 for panel D) |
| Mg²⁺(25 mM) | 0.80 |
| 10X buffer w/o Mg²⁺ | 1.00 |
| (JMR Holding Inc.) | |
| dNTP (25 mM) | 0.10 |
| Taq polymerase (5 μl) | 0.08 |
| (Super Therm Gold Taq) | |
| Primers set (2 pmole/μl) | |
| FAM | 0.08(0.16 for panel-D) |
| HEX | 0.20 |
| TMR | 1.50 |
| Genomic DNA (100 ng/μl) | 2.00 |
| Total volume | 15.00 |

The thermocycling protocol of PCR is initiated at 95° C. for a hot start using SuperTherm Gold DNA Polymerase (JMR Holdings) for 7.5 min, followed by 25 cycles of 30 second at 95° C., 1 min at 59° C., and 1.5 min at 72° C. The reaction mix was then incubated at 72° C. for 8 min and finally stored at 16° C. The PCR products were mixed with deionized water and MegaBACE® ET900-R Rhodamine X (Rox) labeled DNA size standard (Amersham Bioscience). Denatured amplicons were subjected to electrophoresis on a MegaBACE® Long-Read Matrix capillary array with a MegaBACE® genetic analyzer.

—MegaBACE® Capillary Electrophoresis—

PCR products were separated by MegaBACE® Capillary electrophoresis system: the system was pre-run at 44° C. and 10 kV for 5 min. The sample was injected thereafter at a voltage of 3 kV, with an injection time of 45 s; the running voltage was then set back to 10 kV and run for 100 min.

—MIRU-VNTR Analysis—

The PCR products were sized and the various VNTR alleles were assigned by Fragment Profiler version 1.2 software (GE-Amersham Bioscience). The genotype was expressed as a numerical code made up by numbers of tandem repeats in each of the 15 genomic loci. The gathered data were finally entered into the backend software through a Microsoft® Open DataBase Connection (ODBC) function for phylogenetic clustering analysis.

—Computer Analysis—

The MIRU-VNTR profiles were analyzed using Bionumerics® software, version 4.0 (Applied Maths., Kortijk, Belgium). The discriminatory power of individual and combined VNTR loci was assessed through allelic diversity (h), calculated using the equation h=1−ΣXᵢ² {n/(n−1)}, where n is the number of strains and Xi is the frequency of the ith allele at the locus. The HGDI was calculated based on Robin A. S. et al., Microbiology, 2002, 148, 519-528, herein incorporated in its entirety.

—Result—

Allelic diversities of the 15 MIRU-VNTR loci were determined followed by PCRs, electrophoresis and computer analysis mentioned above. It was found that loci 10, 26, and 31 were highly discriminative (≧0.6), loci 4, 16, 23, 39, 40, ETR-A, and ETR-B moderately discriminative (≧0.3), and loci 2, 20, 24, and 27 poorly discriminative (<0.3) (Table 3). The resulting higher polymorphism of the 15-locus scheme attributes to both ETR-A and ETR-B having a medium allelic diversity index of 0.592 and 0.589, respectively (Table 3). In this study, the HGDI for MIRU-VNTR using 12 loci was 0.951; whereas the value for the combined 15 loci reached 0.972 for the M. tuberculosis isolates examined (Table 4). The discriminatory power of the 15-MIRU-VNTR-locus scheme is comparable to that found in previous studies using the 12-locus scheme. The allelic diversity and derived HGDI are highly dependent upon the set of isolates being tested.

The genetic relationships among the 502 isolates were analyze using the unweighted pair group method algorithm (UPGMA). The discriminatory capacity of the 12 or 15 MIRU-VNTR loci resolved 186 and 232 profiles, respectively. The prevalence of Beijing family strains determined by spoligotyping was 44.4% in Taiwan. In this study, 43.6% ( 219/502) of the isolates previously genotyped as Beijing family strains were readily distinguishable from each other by MIRU-VNTR. Sixty-nine and 84 distinct MIRU-VNTR profiles were obtained, respectively, by the 12- and 15-locus schemes. Beijing family isolates were all closely related by genetic distance analysis: 60% and 73% for the 12- and 15-locus schemes, respectively.

TABLE 3
Allelic diversity and discriminatory power of
each locus and 15 combined loci of MIRU-VNTR

h^{a}

| Locus | 1^{b} | 2^{b} | 3^{b} | 4^{b} | 5^{b} | 6^{b} |
|---|---|---|---|---|---|---|
| 2 | 0.02 | 0.08 | 0.241 | 0.14 | 0.20 | 0.084 |
| 4 | 0.35 | 0.22 | 0.479 | 0.28 | 0.50 | 0.316 |
| 10 | 0.69 | 0.44 | 0.617 | 0.70 | 0.71 | 0.659 |
| 16 | 0.52 | 0.42 | 0.526 | 0.28 | 0.31 | 0.309 |
| 20 | 0.29 | 0.09 | 0.205 | 0.06 | 0.03 | 0.058 |
| 23 | 0.58 | 0.12 | 0.656 | 0.54 | 0.42 | 0.343 |
| 24 | 0.24 | 0.16 | 0.445 | 0.00 | 0.35 | 0.199 |
| 26 | 0.67 | 0.54 | 0.688 | 0.59 | 0.73 | 0.770 |
| 27 | 0.19 | 0.09 | 0.124 | 0.14 | 0.21 | 0.166 |
| 31 | 0.37 | 0.47 | 0.647 | 0.55 | 0.64 | 0.702 |
| 39 | 0.34 | 0.22 | 0.394 | 0.36 | 0.60 | 0.541 |
| 40 | 0.74 | 0.63 | 0.797 | 0.65 | 0.54 | 0.475 |
| ETR-A | ND^{c} | ND | 0.756 | ND | ND | 0.592 |
| ETR-B | ND | ND | 0.530 | ND | ND | 0.589 |
| ETR-C | ND | ND | 0.584 | ND | ND | 0.133 |
| HGDI | ND | ND | ND | ND | 0.975 | 0.972 |

^{a}Allelic diversity, h = 1 − ΣXᵢ² {n/(n − 1)} where n is the number of strains and Xi is the frequency of the ith allele at the locus
^{b}1. Mazars et al. 2001. PNAS 98: 1901-1906; 2. Cowan et al. 2002. J. Clin. Microbiol. 40: 1592-1602; 3. C. Sola et al. 2003. Infect. Genet Evolution. 3: 125-133; 4. Supply et al. 2003. Mol. Microbiol. 47: 529-538; 5. Sun et al. 2004. J. Clin. Microbiol. 42: 1986-1993; 6. This study.
^{c}Not determined.

TABLE 4
statistics analysis of MIRU-VNTR using 12 and 15 loci

| | 12 loci | 15 loci |
|---|---|---|
| Sample size | 502 | 502 |
| Cluster number | 186 | 232 |
| HGDI | 0.951 | 0.972 |

Utility

Compare the cost and operation time of the invention with that of Supply P. et al (2001) and Allix (2004).

TABLE 5
comparison of cost¹

| | methods | | |
|---|---|---|---|
| | | | This |
| materials & | | | invention, |
| concentration | Supply, 2001 | Allix, 2004 | 2004 |
| DNA | 0.2 mM | 0.2 mM | 0.25 mM |
| | (Amersham) | (Amersham) | (Amersham) |
| Thermo- | 1 U | 1 U | 0.4 U |
| resistant | (Qiagen | (Qiagen | (JMR Holding) |
| polymerase | HotStart Taq) | HotStart Taq) | |
| Fluorescent | 0.4 μM | 0.4 μM | 0.1˜1.0 μM |
| primer | (Patented | (Patented Dye; | (Public Dye) |
| | Dye; HED) | VIC) | |
| Total reaction | 50 | 50 | 10 |
| volume (μl) | | | |
| Unit cost | ˜1.3 | ˜1.3 | ˜0.6 |
| (USD)² | | | |

Note¹: based on capillary electrophoresis experiment
Note²: roughly estimated by prices of manufacturers in Taiwan area (TWD:USD = 31.15:1)

TABLE 6
PCR reaction comparison

| | | | the |
|---|---|---|---|
| | Supply, | | invention, |
| | 2001 | Allix, 2004 | 2004 |
| 1. Hot start | 15 min | 15 min | 10 min |
| 2. Denature | 1 min | 1 min | 30 s |
| 3. Annealing | 1 min | 1 min | 1 min |
| 4. Elongation | 1 min 30 s | 1 min 30 s | 1 min 30 s |
| 5. Repeated cycle of | 40 | 25 | 25 |
| Step 2.˜Step 4. | | | |
| 6. Termination | 10 min | 10 min | 8 min |
| Total reaction | ˜4 hrs | ˜2 hrs 30 min | ˜2 hrs |
| period(estimate) | | | |

Note ¹: required time of reaction wherein Applied BioSystems 9700 Thermocycler as standard machine

According to the invention, the cost of a unit sample is reduced by 50%. Moreover, labor requirements are decreased compared to RFLP. The discriminatory power is equal to or higher than conventional MIRU-VNTR genotyping (Supply et al., 2001, J. Clin. Microbiol. 39:3563-3571). Because PCR and capillary electrophoresis of this method are automated, therefore, artificial errors can be avoided and consequently, easily to be reproduced.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation to encompass all such modifications and similar arrangements.

1. An automated VNTR genotyping method comprising:
obtaining a DNA fragment from a sample of interest,
amplifying the DNA fragment by a PCR reaction,
detecting the amplified DNA fragment by an automated system,
wherein the PCR reaction uses primers labeled by fluorescent dyes selected from the group consisting of TAMRA, FAM and HEX.

2. The automated VNTR genotyping method as claimed in claim 1, further comprising a capillary electrophoresis system.

3. The automated VNTR genotyping method as claimed in claim 2, wherein the capillary electrophoresis system is MegaBACE® capillary electrophoresis system.

4. The automated VNTR genotyping method as claimed in claim 1, wherein the sample is from microorganisms, parasites, animals, or plants.

5. The automated VNTR genotyping method as claimed in claim 4, wherein the microorganism is mycobacteria.

6. The automated VNTR genotyping method as claimed in claim 5, wherein the microorganism is Mycobacterium tuberculosis.

7. The automated VNTR genotyping method as claimed in claim 1, wherein the primers are correspondent to loci of VNTR listed in the following:

| | | |
|---|---|---|
| | VNTR | |
| | Length | Primer sequence |
| Locus^{a} | (bp) | (labeling) (5′-3′)^{b} |

SEQ ID NOS: 1-15.

| | | |
|---|---|---|
| 4 | 77 | (FAM)GCGCGAGAGCCCGAACTGC |
| (ETR-D) | | GCGCAGCAGAAACGCCAGC |
| 26 | 51 | TAGGTCTACCGTCGAAATCTGTGAC |
| | | (HEX)CATAGGCGACCAGGCGAATAG |
| 40 | 54 | (TAMRA)GGGTTGCTGGATGACAACGTGT |
| | | GGGTGATCTCGGCGAAATCAGATA |
| 10 | 53 | GTTCTTGACCAACTGCAGTCGTCC |
| | | (FAM)GCCACCTTGGTGATCAGCTACCT |
| 16 | 53 | TCGGTGATCGGGTCCAGTCCAAGTA |
| | | (HEX)CCCGTCGTGCAGCCCTGGTAC |
| 31 | 53 | ACTGATTGGCTTCATACGGCTTTA |
| (ETR-E) | | (TAMRA)GTGCCGACGTGGTCTTGAT |
| 2 | 53 | TGGACTTGCAGCAATGGACCAACT |
| | | (FAM)TACTCGGACGCCGGCTCAAAAT |
| 23 | 53 | (HEX)CTGTCGATGGCCGCAACAAAACG |
| | | AGCTCAACGGGTTCGCCCTTTTGTC |
| 39 | 53 | CGCATCGACAAACTGGAGCCAAAC |
| | | (TAMRA)CGGAAACGTCTACGCCCCACACAT |
| 20 | 77 | (FAM)TCGGAGAGATGCCCTTCGAGTTAG |
| | | GGAGACCGCGACCAGGTACTTGTA |
| 24 | 54 | CGACCAAGATGTGCAGGAATACAT |
| | | (HEX)GGGCGAGTTGAGCTCACAGAA |
| 27 | 53 | TCGAAAGCCTCTGCGTGCCAGTAA |
| | | (TAMRA)GCGATGTGAGCGTGCCACTCAA |
| ETR-A | 75 | (FAM)AAATCGGTCCCATCACCTTCTTAT |
| | | CGAAGCCTGGGGTGCCCGCGATTT |
| ETR-B | 57 | (HEX)GCGAACACCAGGACAGCATCATG |
| | | GGCATGCCGGTGATCGAGTGG |
| ETR-C | 58 | GTGAGTCGCTGCAGAACCTGCAG |
| | | (TAMRA)GGCGTCTTGACCTCCACGAGTG |


SEQUENCE LISTING (16 sequences)

SEQ ID NO: 1 - DNA, 38 bp, Artificial Sequence
  Oligonucleotide primer for multiplex PCR of MIRU-VNTR analysis
gcgcgagagcccgaactgcgcgcagcagaaacgccagc

SEQ ID NO: 2 - DNA, 46 bp, Artificial Sequence
  Oligonucleotide primer for multiplex PCR of MIRU-VNTR analysis
taggtctaccgtcgaaatctgtgaccataggcgaccaggcgaatag

SEQ ID NO: 3 - DNA, 46 bp, Artificial Sequence
  Oligonucleotide primer for multiplex PCR of MIRU-VNTR analysis
gggttgctggatgacaacgtgtgggtgatctcggcgaaatcagata

SEQ ID NO: 4 - DNA, 47 bp, Artificial Sequence
  Oligonucleotide primer for multiplex PCR of MIRU-VNTR analysis
gttcttgaccaactgcagtcgtccgccaccttggtgatcagctacct

SEQ ID NO: 5 - DNA, 46 bp, Artificial Sequence
  Oligonucleotide primer for multiplex PCR of MIRU-VNTR analysis
tcggtgatcgggtccagtccaagtacccgtcgtgcagccctggtac

SEQ ID NO: 6 - DNA, 43 bp, Artificial Sequence
  Oligonucleotide primer for multiplex PCR of MIRU-VNTR analysis
actgattggcttcatacggctttagtgccgacgtggtcttgat

SEQ ID NO: 7 - DNA, 46 bp, Artificial Sequence
  Oligonucleotide primer for multiplex PCR of MIRU-VNTR analysis
tggacttgcagcaatggaccaacttactcggacgccggctcaaaat

SEQ ID NO: 8 - DNA, 48 bp, Artificial Sequence
  Oligonucleotide primer for multiplex PCR of MIRU-VNTR analysis
ctgtcgatggccgcaacaaaacgagctcaacgggttcgcccttttgtc

SEQ ID NO: 9 - DNA, 48 bp, Artificial Sequence
  Oligonucleotide primer for multiplex PCR of MIRU-VNTR analysis
cgcatcgacaaactggagccaaaccggaaacgtctacgccccacacat

SEQ ID NO: 10 - DNA, 48 bp, Artificial Sequence
  Oligonucleotide primer for multiplex PCR of MIRU-VNTR analysis
tcggagagatgcccttcgagttagggagaccgcgaccaggtacttgta

SEQ ID NO: 11 - DNA, 45 bp, Artificial Sequence
  Oligonucleotide primer for multiplex PCR of MIRU-VNTR analysis
cgaccaagatgtgcaggaatacatgggcgagttgagctcacagaa

SEQ ID NO: 12 - DNA, 46 bp, Artificial Sequence
  Oligonucleotide primer for multiplex PCR of MIRU-VNTR analysis
tcgaaagcctctgcgtgccagtaagcgatgtgagcgtgccactcaa

SEQ ID NO: 13 - DNA, 48 bp, Artificial Sequence
  Oligonucleotide primer for multiplex PCR of MIRU-VNTR analysis
aaatcggtcccatcaccttcttatcgaagcctggggtgcccgcgattt

SEQ ID NO: 14 - DNA, 44 bp, Artificial Sequence
  Oligonucleotide primer for multiplex PCR of MIRU-VNTR analysis
gcgaacaccaggacagcatcatgggcatgccggtgatcgagtgg

SEQ ID NO: 15 - DNA, 45 bp, Artificial Sequence
  Oligonucleotide primer for multiplex PCR of MIRU-VNTR analysis
gtgagtcgctgcagaacctgcagggcgtcttgacctccacgagtg

SEQ ID NO: 16 - DNA, 18 bp, Artificial Sequence
  Oligonucleotide primer for multiplex PCR of MIRU-VNTR analysis
gcgcagcagaaacgccag